(19) United States
(12) Patent Application Publication
(10) Pub. No.: US 2004/0235189 A1
(43) Pub. Date: Nov. 25, 2004

(54) Reversed chromatographic immunoassay

(76) Inventor: Wei Zhao Lu, San Diego, CA (US)

Correspondence Address: Wei Zhao Lu, LuSys Laboratories, Inc., 3716 Carmel View Road, San Diego, CA, 92130, US

(21) Appl. No.: 10/835,486

(22) Filed: Apr. 30, 2004

Related U.S. Application Data
(60) Provisional application No. 60/469,296, filed on May 8, 2003.

(51) Int. Cl. G01N 33/558

(52) U.S. Cl. 436/514; 435/970; 435/287.2

(57) ABSTRACT

A chromatographic immunoassay test strip comprising of a solid support having the portions with said portions being in a strip so as to permit capillary flow communication with each other based on the help of adding a Buffer. This chromatographic immunoassay test strip herein changes the reaction order of the conventional chromatographic immunoassay solid test strip, wherein the analyte in the sample reacts with the specific binder (ligand A) immobilized on test zone prior to reacting with the ligand B/tracer. The reaction communication herein is achieved by a capillary flow aided by a Buffer. This solid chromatographic immunoassay test strip is useful in a variety of immunoassays.

### BACKGROUND OF INVENTION

This invention relates to the chromatographic immunoassay test strip, and more particularly to a sequential mode, i.e. a chromatographic immunoassay test strip with the sample adding point being placed in close to the binder region and in front of the solid phase ligand B/tracer so that allowing the said analyte in the sample reacts with the specific binder immobilized at testing region prior to any other reaction taking place. But the capillary flow is achieved by use of a Buffer flow.

Assays for various analytes have been accomplished by a solid phase assay. Chromatographic immunoassay is as generally known in the art. U.S. Pat. No. 3,011,874 discloses a test strip representative of this type (FIG. 1). In such assay, a solid support has five portions and the portions being in capillary flow communication with each other whereby material flows by capillarity. The first and second portions are positioned on the solid support in a manner such that the first portion may be contacted with the material, including any analyte, with material in said first portion being transported by capillarity from the first portion of the support to the second portion thereof and so on. The second portion of the solid support includes a ligand which is able to react with at least the analyte. The second portion comprised of a ligand portion and a detectable label portion conjugated to the ligand B portion as a tracer. In the case of where the assay format is an immunology sandwich assay format, the ligand B/tracer portion is bound with the analyte first. The ligand B/tracer is supported on the solid support in a manner such that when wetted, the ligand B/tracer is capable of being transported by capillarity to other portion of the solid support. The specific binder to the analyte is immobilized on 3^{rd} portion said test region. In the case of where the lateral flow is in the process, the “ligandB/tracer-analyte” complex will be bound on the test region on which a “ligand A—analyte-ligand B/tracer” complex will be formed. Thereafter, depending on the presence and/or absence of analyte and/or the amount of analyte, a T line might be detectable with or without equipment.

There are limitations by using chromatographic immunoassay mentioned above. For example, specific IgG/IgM to a certain antigen in human serum specimen which contains both specific (very small portion in the specimen) and nonspecific IgG/IgM (large portion in the specimen), competitively react with the ligand B/tracer, such as anti-human IgG/IgM (2^{nd} antibody) labeled with a dye in aforesaid assay. When the conventional chromatographic immunoassay method is employed, most anti-human IgG/IgM (2^{nd} antibody) labels have reacted and been neutralized by the non-specific human IgG/IgM in the specimen before they reached the immobilized binder (“T line”). The complex of “ligand A—analyte-ligand B/tracer” cannot be formed or if so very little will be formed on the T line. So there is either none or a very faint T line that will be detectable. Therefore, the conventional chromatographic immunoassay might not be used for certain tests. In a particularly preferred embodiment, chromatographic immunoassay might not be used at detecting the human antibody specific to infectious antigen and/or auto antibodies in a human.

### SUMMARY OF THE INVENTION

The purpose of the present invention is to create a new format of the chromatographic immunoassay. The present invention is directed to providing an improved solid phase assay for determining certain analyte, and more particularly a broader assay spectrum by changing what is known in the art The aforementioned solid chromatographic immunoassay assay reaction order is reversed and communication achieved by the placement of the sample and the addition of a buffer (FIG. 2).

The application of present invention can be used to obtain a single test data or multiple-test data in one test device.

The present invention overcomes the aforesaid shortcomings by changing and using the said test strip in a novel way. The sample region is placed in front of the ligand B/tracer region and just behind/at the “T” line. A Buffer is introduced to the test strip where the sample is added in the conventional test. This will facilitate the movement of the sample along the strip and allow it to react with the binder immobilized in the test region further and then allowing the ligand B/tracer to follow and react with the said analyte/ligand A which already have been formed on the test line.

In accordance with the present invention, there is provided a solid support having a first portion for adding the buffer, and a second portion for immobilizing ligand/tracer, a third portion for adding small amount of specimen, a fourth portion for the binder determining the characteristics of the analyte in the specimen which the complex band that will be detectable will be formed herein depending on the presence of analyte in the specimen, a fifth portion for control, and a sixth portion for absorbing liquid traveling through the porous materials from the liquid flow.

The solid support employed in the assay which provides the capillary flow paths for the ligand B/tracer, specimen, and buffer are in the one strip. The solid support also provides a surface area capable of supporting the binder—ligand A. As examples, of such materials, there may be but not limited to: glass fiber, cellulose, nylon, various chromatographic paper, nitrocellulose, etc. The solid support is preferably in the sheet form, generally being in the form of a card, a strip or dipstick, etc.

The type of the binder employed in the assay which is immobilized may be an antigen, a specific protein, or antibodies. If it is an assay for determining an antibody, then the binder may be antigen or a specific protein or an antibody which is specific for the antibody to be assayed. If the analyte is an antigen or a specific protein, then the binder may be antibody or other materials which is specific for the antigen to be assayed.

The ligand B which is labeled for use as the tracer in the assay is dependent upon the analyte to be assayed. The ligand B/tracer would be bound to the analyte or/and bound to non-specific component in the specimen which will flow pass the solid support aided by the Buffer. To produce the tracer, the ligand may be labeled with a particular label as a detectable marker. The particular label may be visible includes but is not limited to a dye or any colored substance, such as gold particle, colored latex, liposome, erythrocytes, polymer particles, bacterial and other materials. The particulate label may be non-solid labels, such as radiotopes, enzymes, fluorescent compounds or other chromogen labels, dyes or chemiluminescent materials that either produce or catalyze a color-developing reaction which may be detected with or without further treatment and with or without the use of instrumentation. The Buffer in the assayed herein are general chemical buffers, such as phosphate buffer, Tris buffer and even the distilled water. The selection of a suitable buffer is deemed to be the skill in art. With the aid of buffer, the capillary flow on the solid support may be happened. With the aid of buffer, the analyte may be helped to react further with the binder on Test region prior to arrival of the tracer while the ligand/tracer is wetting. With the aid of buffer, the all immuno reaction communication on the same plane may be performed and the results that may be achieved include wetting the ligand B/tracer and introducing it to pass the binder which might form a “lignad A—analyte-ligand B/tracer” complex on the test region.

The procedure employed herein which is capable of absorbing analyte from the sample. By adding the buffer, the ligand B/tracer are dissolved and which, when wetted, provides for the flow of analyte and ligand B/tracer by capillary attraction from the first portion to the other portion of the solid support. In addition, the solid support is one which is capable of supporting the ligand B/tracer and the ligand A—binder. Porous capillarity-possessing materials are suitable for use as solid support, such as glass fiber, nylon, chromatographic papers, nitrocellulose, etc.

The test line binder—ligand A is immobilized on a solid support in an appropriate concentration, as herein above described, is initially contacted with analyte. For example, the binder is an antigen and the analyte is an antibody. Subsequently, with or without a blocking procedure on the same area by adding a block reagent, the antibody will be bound to the binder on the solid support in the test region. The ligand B/tracer is anti-antibody to the analyte (2nd antibody) labeled with a particulate label, such as a gold particles. The amount of ligand B/tracer which is bound to the binder on the solid support through the analyte is directly proportional to the amount of analyte in the sample, and the presence and/or amount of analyte present in the sample may be determined from the presence and/or amount of tracer which becomes bound to the support through the analyte.

The present invention is applicable to detecting a wide variety of analytes, such as: TORCH antibodies, other antibodies induced by invasion or said infectious antigen, auto-antibodies in human or animals, etc.

### BRIEF DESCRIPTION OF THE DRAWING

FIG. 1 is a schematic representation of a conventional chromatographic test strip, wherein “s” represents the first portion of the test strip, which is sample adding area; “Conj.” represents the second portion of the test strip, in said portion a ligand B labeled/tracer, such as a gold conjugate. The “ligand B/tracer-analyte” may be formed if the analytes in the said sample present; “T” represent a third portion, in said portion the binder(ligand A) immobilized which will react with the “ligand B labeled/tracer-analyte” to form “ligandA—ananlyte-ligandB/tracer-analyte” complex when the communication be achieved. A color band will appear on T region if the analytes in the said sample present after a period of time; “C” represent a forth portion, wherein a non-specific binder is immobilized; and “Abs” represent a fifth portion of the test strip, wherein a bibulous material is employed.

FIG. 2 is a schematic representation of a reversed chromatographic test strip, wherein “B” represent the first portion of the test strip, which is buffer adding well; “Conj.” represents the second portion of the test strip, in said portion a ligand B/tracer, such as a gold conjugate of ligand B; “s” represents the third portion of the test strip, which is sample adding area; “T” represent a forth portion, in said portion the binder immobilized will react with the analyte in the sample to form “ligand A—analyte” complex and then form the “lignad A—analyte-ligand B labeled/tracer” complex sequentially when the communication be achieved by a Buffer flow; “C” represent a fifth portion, wherein a non-specific binder is immobilized; and “Abs” represent a sixth portion of the test strip, wherein a bibulous material is employed.

FIG. 3 is a schematic representation of a Five-in-One TORCH diagnosis kit using reversed chromatographic test strip, wherein “B” represent the first portion of the test strip, which is buffer adding well; “Conj.” represents the second portion of the test strip, in said portion, a ligand B/tracer, such as, a gold conjugate of mouse antihuman IgG/M; “s” represents the third portion of the test strip, which is sample adding area; “T” represent a forth portion, in said portion the binder (ligand A) immobilized, such herein HSV-lantigen, HSV-antigen II, Rubella antigen, CMV antigen, Toxoplasma antigen immobilized separately, will react with the analyte in the sample to form “ligand A—analyte” complex and then form the “lignad A—analyte-ligand B/tracer” complex sequentially when the communication be achieved by a Buffer flow; “C” represent a fifth portion, wherein a non-specific binder in accordance with the designed, such as goat anti-mouse IgG antibody, and “Abs” represent a sixth portion of the test strip, where the bibulous materials are employed.

The present invention will now be illustrated, but is not intended to be limited, by the following Example.

### EXAMPLE

### I) Single Reversed Chromatographic Immunoassay Torch Test Cassette

A. Materials Needed

1) 10 mil Matte vinyl board with adhesive on one side (G & L, San Jose, Calif.)

2) nitrocellulose membranes (Saitorius, Edgewood, N.Y.)

3) bibulous paper (Whatman, Fairfield, N.J.)

4) glass fiber (Pall-Gelman, Ann Arbor, Mich.)

5) gold chloride (Sigma, St. Louis, Mo.)

6) TORCH antigens: Toxoplasma (call Toxo), Cytomegalovirus (call CMV), Rubella, Herpes Simplex Virus-I (call HSV-I), Herpes Simplex Virus II(call HSV-II) (Ross Southern Lab, Utah)

7) mouse anti-human IgG/IgM (Biopacific, Calif.)

8) goat anti-mouse IgG (E & E Labs, S. San Francisco, Calif.)

B. Preparation of Reversed Chromatographic Immunoassay Test Strip

1) using reagent dispenser to dispense 1-2 mg/ml TORCH (Toxo, CMV, Rubella, HSV-I and HSV-II) antigens separately on 25 mm*300 mm independent nitrocellulose membranes to make Test line;

2) using reagent dispenser to dispense 2 mg/ml goat anti-mouse IgG antibodies on 25 mm*300 mm nitrocellulose membranes to make Control line;

3) marking 10 ml gold conjugate with 25 μg mouse anti-human IgG or IgM antibodies (Chinese patent document 98122892.5);

for IgG assay, using mouse anti-human IgG to make the label ligand;

for IgM assay, using mouse anti-human IgM to make the ligand B labeled;

4) using centrifuge at 12,000 rpm speed to separate the gold conjugate—ligand B/tracer;

5) immersing glass fiber in liquid of gold conjugate, which is dissolved by a kind of phosphate buffer, dried by a vacuum dryer and cut it into 6*30 mm strips;

C. Assembling the Test Kit

1) affixing the glass fiber; gold conjugate; nitrocellulose membrane bound with an antigen as T line and bound with goat anti-mouse IgG as Control line, and bibulous paper on the solid support in a juxtapositioned relationship and cut to 6 mm test strip;

2) affixing the aforementioned the different test strips into a plastic cassette.

### II) Five-in-one Reversed Chromatographic Immunoassay Torch Test Cassette

A. Materials Needed as the Same as Described Above.

B. Preparation of Reversed Chromatographic Immunoassay Test Strip as the Same as Described Above.

C. Assembling the Five Different Strips Into Five-in-one Cassette.

### II) A Comparison Stusy of Enzyme Immunoassay (ELISA), Conventional Chromatographic Immunoassay (CCI), and Reversed Chromatographic Immunoassay (RCI)

A. Collect Samples

1) 72 patients serum samples are used to test for Toxo antibodies. 38 of those said samples are used to test for IgG antibodies. 34 of those said samples are used to test for IgM antibodies.

2) 81 patients serum samples are used to test for CMV antibodies. 42 of said samples are used to test for IgG antibodies. 39 of said samples are used to test for IgM antibodies.

3) 68 patients serum samples are used to test for Rubella antibodies. 36 of said samples are used to test for IgG antibodies. 32 of said samples are used to test for IgM antibodies.

4) 120 patients serum samples are used to test for HSV-I antibodies. 64 of said samples are used to test for IgG antibodies. 56 of said samples are used to test for IgM antibodies.

5) 123 patients serum samples are used to test for HSV-I antibodies. 70 of said samples are used to test for IgG antibodies. 53 of said samples are used to test for IgM antibodies.

B. ELISA TORCH Antibodies Test Kit Manufactured by E&E Labs, CA

C. Reversed Chromatographic Immunoassay Test Kit

D. Conventional Chromatographic Immunoassay Test Kit

E. Test:

1) testing the said samples for Toxo, CMV, Rubella, HSV-I, and HSV-II antibodies separately using ELISA TORCH test kits according to the instruction of the used products. The said sample has positive reaction when OD>0.6. The said sample has negative reaction when OD<0.25.

2) testing the said samples for Toxo, CMV, Rubella, HSV-I, and HSV-II antibodies separately using reversed chromatographic immunoassay test kits.

a) adding 5 μl said serum sample at the sample adding point as showed in FIG. 3.

b) adding two drops of buffer at the buffer adding area as showed in FIG. 3.

c) waiting for 15 minutes. The said sample has positive reaction when a pink band showed at both Test kine and Control line as showed in FIG. 3. The said sample has negative reaction when a pink band only showed at Control line.

3) testing the said samples for Toxo, CMV, Rubella, HSV-I, and HSV-II antibodies separately using conventional chromatographic immunoassay test kits.

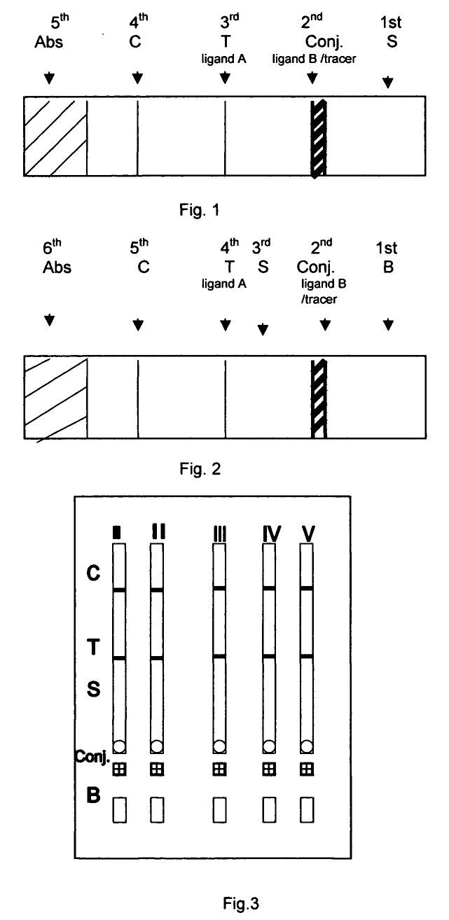

a) adding 80 μl said serum sample at the sample adding point as showed in FIG. 1.

b) waiting 15 minutes. The said sample has positive reaction when a pink band showed at both Test line and Control line as showed in FIG. 2. The said sample has negative reaction when a pink band only showed at Control line.

F. Test Results

Test Results Data Listed in Table 1.

The test result shows that using conventional chromatographic immunoassay test kits to test the said all positive patients serum samples for TORCH antibodies obtaining 100% negative results. On the other hand, the correlation of the positive results between using ELISA and reversed chromatographic immunoassay is greater than 94%.

TABLE 1

| | | | | | CORRELATION |
|---|---|---|---|---|---|
| | | | | | BETWEEN |
| | | ELISA | RCI | CCI | ELISA AND RCI |
| TOXO | | | | | |
| IgG | Negative | 14 | 14 | 14 | 100% |
| | Positive | 24 | 23 | 0 | 96% |
| IgM | Negative | 16 | 16 | 16 | 100% |
| | Positive | 18 | 17 | 0 | 94% |
| CMV | | | | | |
| IgG | Negative | 18 | 18 | 18 | 100% |
| | Positive | 24 | 23 | 0 | 96% |
| IgM | Negative | 22 | 22 | 22 | 100% |
| | Positive | 17 | 16 | 0 | 94% |
| Rubella | | | | | |
| IgG | Negative | 12 | 12 | 12 | 100% |
| | Positive | 24 | 23 | 0 | 96% |
| IgM | Negative | 11 | 11 | 11 | 100% |
| | Positive | 21 | 20 | 0 | 95% |
| HSV-1 | | | | | |
| IgG | Negative | 18 | 18 | 18 | 100% |
| | Positive | 46 | 45 | 0 | 98% |
| IgM | Negative | 21 | 21 | 21 | 100% |
| | Positive | 35 | 34 | 0 | 97% |
| HSV-2 | | | | | |
| IgG | Negative | 16 | 16 | 16 | 100% |
| | Positive | 54 | 53 | 0 | 98% |
| IgM | Negative | 19 | 19 | 19 | 100% |
| | Positive | 34 | 33 | 0 | 97% |

I claim:

1. A reversed chromatographic immunoassay test strip for the detection of an ingredient of a sample which comprises a matrix, said matrix containing:
(A) 1^{st} region: a buffer adding region having a porous support permitting liquid travel through the solid phase; and
(B) 2^{nd} region: a solid phase ligand B/tracer being placed on the porous support; and
(C) 3^{rd} region: a sample adding region having the location just in front of the ligand/tracer region and behind/at the test region—the binder region said a test region .
(D) 4^{th} region: a test region (call T line) having bound and immobilized the binder—ligand A which are to react with specific analytes if present in the sample to form a specific binder-analytes complex first, then will react with the dissolved ligand/tracer to form a “binder-analyte-ligand/tracer” complex which will be detectable; and
(E) 5^{th} region: a control region containing materials capable of reacting with said non-specific materials to produce a color forming reaction which indicates the presence of said ligand/tracer; and
(F) 6^{th} region: a bibulous material region for helping the lateral flow process. E & F.

2. A reversed chromatographic immunoassay method wherein a sample is first introduced to the test strip just in front of the ligand B/tracer region and behind/at the region of the binder—ligand A region (Call “T line”). The analytes if presents in the sample will react with the ligand A partially wherein. Secondly a buffer will be introduced to the test strip in the 1^{st} region. The buffer will accomplish two goals, (a) by capillary flow it will move along the test strip and aid the sample in moving toward the test region wherein the specific analytes if contained in the sample will react further with the binder (ligand A), which is immobilized in the test region, (b) release of the ligand B/tracer and allowing it to travel along the test strip toward the test line where the ligand B will bind with the ligand A—analyte complex already formed if said analytes are contained in the sample, forming a ligand A—analyte-ligand B complex.

3. An application of said test strip mentioned in claim 1 and claim 2 for detection of Torch (i.e. Toxoplasma gondii, Cytomegalovirus, Rubella, and Herpes Simplex virus I & II) antibodies assay, but not limited to this application since there are other applications using this procedure for detection of antibodies and/or antigens but work along the same principle as said application.
